(19) United States
(12) Patent Application Publication
(10) Pub. No.: US 2024/0272087 A1
(43) Pub. Date: Aug. 15, 2024

(54) CONTAINER INSPECTION SYSTEM

(71) Applicants: Liraz SHLOMOFF, Rehovot (IL); Ofer VIKINSKY, Tzur Yigal (IL); Eyal ZYLBERBERG, Kfar Yona (IL)

(72) Inventors: Liraz SHLOMOFF, Rehovot (IL); Ofer VIKINSKY, Tzur Yigal (IL); Eyal ZYLBERBERG, Kfar Yona (IL)

(21) Appl. No.: 18/681,060

(22) PCT Filed: Aug. 7, 2022
(86) PCT No.: PCT/IL2022/050857

§ 371 Date: Feb. 4, 2024

Related U.S. Application Data
(60) Provisional application No. 63/230,789, filed on Aug. 8, 2021.

(51) Int. Cl. G01N 21/90

(52) U.S. Cl. CPC G01N 21/9072; G01N 21/909

(57) ABSTRACT

The present invention is of a system for inspection of containers, the system comprising: a chamber for accommodating a container, the chamber unlit and sealed to any external light; at least one light source configured to be positioned in the container to illuminate the interior of the container; and at least one light detection device disposed at the interior of the chamber and configured to detect light from the at least one light source, wherein the detected light is emitted from the container into the chamber; wherein the detected light is used to detect damage to the container.

### FIELD OF THE INVENTION

The present invention relates to a detection system. Moreover, the present invention is of a system for detecting damage to a container.

### BACKGROUND OF THE INVENTION

Typically, surgical instruments which have been sterilized may be stored in a sterilization container, which is designed to maintain a sterile environment before use of the instruments. The containers may allow flow of steam from an autoclave into the container during sterilization and prevent non sterile air accessing the container during storage. Damage to any component of the container can allow penetration of pathogens and contamination of the contents of the container. Contaminated instruments increase the risk of surgical site infections (SSI). As such, it is important to inspect sterilization containers for any damage, which could affect the containers and result in contamination of previously sterile equipment.

A standard for inspection of these type of containers is usually done by certified laboratories, which test for residues of contaminants after sterilization and after a certain shelf life. This type of test is expensive, lengthy, and in many cases cannot be done routinely in a hospital or other medical environment.

A method commonly used is visual inspection, which is done at the same time as packing sterilization containers with instruments to be sterilized. A packer may examine the fit of the lid and external defects in the body and sealing lip. However, this method is limited as it is based on a subjective impression of the functional integrity of the container. Smaller defects may be missed as they may not be easily evident to the eye. Only about fifty percent of the defects are found using this flawed technique.

It would therefore be desirable to have a method which can effectively detect most if not all mechanical defects in a sterilization container. It would be further desirable if the method is reproducible and controlled for producing a standard result. It would also be advantageous if the method could be done during the packaging process in the medical facility. Moreover, it would be better to have a method which does not leave deposits (gas, or fluid residuals) on the contents of the sterilization container. In addition, it would be beneficial if the method could be used to create a performance grade for sterilization containers. The present invention provides such a system and methods of use thereof.

### SUMMARY

The invention may have several aspects.

In one aspect, the invention provides a system for inspection of containers, the system comprising:
a chamber for accommodating a container,
at least one energy source configured to be positioned in the container; and
at least one energy detection device disposed at the interior of the chamber and configured to detect energy from the at least one energy source, wherein the detected energy is emitted from the container into the chamber; wherein the detected energy is used to detect damage to the container.

In one or more embodiments, the energy source is selected from a light source, a heat source, electromagnetic radiation (e.g., radio waves), and sound waves (e.g., ultrasound).

In one or more embodiments, the energy detection device is selected from a light detector, a heat detector, a sound detector and a radiation detector.

In one or more embodiments, the energy source is a light source, and the energy detection device is a light detection device.

In one or more embodiments, the energy source is a heat source, and the energy detection device is a heat detection device/heat detector. In one or more embodiments, the energy source is a heat source, the energy detection device is a heat detection device/heat detector, and the container is made from a thermally insulated material.

In one or more embodiments, the energy source is a sound source, and the energy detection device is a sound detection device/sound detector.

In one or more embodiments, the energy source is an electromagnetic radiation source, and the energy detection device is a radiation detection device/detector.

In one aspect, the invention provides a system for inspection of containers, the system comprising:
a chamber for accommodating a container, the chamber unlit and sealed to any external light;
at least one light source configured to be positioned in the container to illuminate the interior of the container; and
at least one light detection device disposed at the interior of the chamber and configured to detect light from the at least one light source, wherein the detected light is emitted from the container into the chamber; wherein the detected light is used to detect damage to the container.

In one or more embodiments, the container is a sterilization container made from an opaque material, the sterilization container comprises a body comprising a cavity for holding an item to be sterilized; a lid for covering the cavity; and at least one inlet for allowing flow of a sterilant into the container, the inlet is optionally covered with a filter or valve.

In one or more embodiments, the at least one light detection device is at least one camera configured to view the container.

In one or more embodiments, the system further comprises a sealing component for sealing the inlet of the sterilization container.

In one or more embodiments, the sealing component is an opaque foil, which covers the inlet.

In one or more embodiments, the system comprises a computer to control the system.

In one or more embodiments, the computer comprises software to provide a score of the sealing capability of the container.

In one or more embodiments, the computer is connected wirelessly to control the at least one light detection device and/or the at least one light source.

In one or more embodiments, the computer commands the at least one light source to perform a sequence of operations.

In one or more embodiments, the sequence of operations comprises at least one of turning the at least one light source on, turning the at least one light source off and changing the color of the light from the at least one light source.

In one or more embodiments, the light is measured in the chamber when the at least one light source is turned on and when it is turned off.

In one or more embodiments, the at least one camera shows the position of where the container is damaged.

In one or more embodiments, the at least one light source is battery operated and is controlled by the system for at least one of sufficient power, intensity and stable lux values.

In one or more embodiments, the at least one inlet with a filter is positioned on the lid of the sterilization container.

In one or more embodiments, when the container is optimally sealed there is at least one of no light illumination, light illumination below a predetermined standard level from the container into the chamber, no detection of light by the at least one light detection device and detection of light below a predetermined level.

In one or more embodiments, when the sealing of the container is damaged, light is radiated from the container into the chamber and detected by the at least one camera.

In one or more embodiments, the computer calculates the level of leakage of light from the container into the chamber.

In one or more embodiments, the computer assigns a usability grade to the container according to the level of leakage of light.

In one or more embodiments, the usability grade comprises a percentage of the perimeter of a detected area of light leakage to the total perimeter of the container or a portion thereof.

In one or more embodiments, the container is approved or disapproved for use according to the usability grade.

In one or more embodiments, the system comprises a platform on which the container is placed, wherein the platform is optimally positioned in relation to the at least one light detection device for detecting of the light emitted from the container into the chamber.

In one or more embodiments, the container comprises a lid that covers the opening of the container and wherein the light detection device detects light emitted from the perimeter of opening of the container.

In another aspect, the present invention provides a method of inspecting containers, the method comprising;
providing a system as herein described;
placing at least one light source in the container, wherein the container is made from an opaque material and comprises a body comprising a cavity for holding an item; and a lid for covering the cavity;
closing the container;
placing the container in the chamber;
closing the chamber;
activating the at least one light source; and
measuring light emitted from the container into the chamber.

In one or more embodiments, the container is a sterilization container.

In one or more embodiments, the sterilization container comprises at least one inlet covered with a filter or valve and wherein the method comprises a step of replacing the filter or valve or covering the filter or valve of the inlet of the container with a sealing component.

In one or more embodiments, activating the at least one light source comprises activating the light source to ascertain the system is working.

In one or more embodiments, activating the at least one light source comprises turning the light source on.

In one or more embodiments, measuring light emitted from the container further comprises calculating the level the container is sealed according to the measured light.

In one or more embodiments, the method comprises calculating a usability grade, wherein the usability grade is a percentage of the perimeter of a detected area of light leakage to the total perimeter of the container or a portion thereof.

In one or more embodiments, the method further comprises approving the container for use according to the level the sterilization container is sealed.

In one or more embodiments, the method further comprises removing the container from the chamber.

In one or more embodiments, removing the container comprises removing the at least one light source from the container.

In one or more embodiments, inspecting sterilization containers is done at the same place the sterilization containers are packed with instruments for sterilization.

The present invention further provides a method of creating a performance record for a container, comprising:
collecting data relating to a container; and
storing the data in a database.

In one or more embodiments, the container is a sterilization container.

In one or more embodiments, the data comprises at least one of the level the sterilization container is sealed, the time the sterilization container has been stored, the number of times the sterilization container has been sterilized, infection of a patient using instruments stored in the sterilization container and time limit factors associated with the sterilization container.

### BRIEF DESCRIPTION OF THE DRAWINGS

The various features of the invention will best be appreciated by simultaneous reference to the description which follows and the accompanying drawings, which are not drawn to scale and in which:

FIGS. 1a, 1b and 1c show perspective views of an exemplary sterilization container;

FIG. 2a shows a side view of an exemplary detection system according to an aspect of the present invention;

FIG. 2b shows a side view of an exemplary lid of a container according to an aspect

of the present invention;

FIG. 3 shows a perspective view of an exemplary sealing component according to an aspect of the present invention;

FIGS. 4a-4c show exemplary images of containers produced using the system of the present invention according to an aspect of the present invention;

FIG. 5 shows a flow chart of an exemplary method of use according to an aspect of the present invention;

FIG. 6 shows a flow chart of an exemplary method according to an aspect of the present invention;

FIG. 7 shows an exemplary table of the shelf-life estimation of a container according to an aspect of the present invention; and

FIGS. 8a and 8b show an exemplary segment of a container captured by a camera according to an aspect of the present invention.

### DETAILED DESCRIPTION

In one aspect the present invention is of a detection system. The detection system is configured to detect the extent of sealing in a container. The container may be a sterilization container. In some embodiments, the container may not be a sterilization container, but may be a container for sealed storing of a material sensitive to the external environment, such as but not limited to light and water sensitive materials. In some embodiments, the container may be for sealed storing of a material or object, which is detrimental to the external environment, such as a toxic substance. In a further aspect, the present invention provides a method of inspecting a container and detecting damage to the sealing capability of the container, such as, but not limited to a sterilization container. Furthermore, the present invention provides a method of creating a performance grade for containers, such as, but not limited to sterilization containers. For example, the invention may be useful to monitor the duration of storage of an inspected container without contamination, and/or occurrence of adverse events (e.g., defects to the container as a result of fall or hit) that result or cause infections.

The detection system and method of use thereof of the present invention have many advantages. The detection system can be used at the site of packing sterilization containers during the packing process. This ensures that there is no delay between use of the container and the sterility approval resulting from the process. The greater the time period between the inspection and use of the container, the greater the chance that the result may no longer be relevant. The system produces reliable reproducible results. Moreover, the human error associated with visual inspection by a worker is avoided. The system is quick and easy to use. In addition, the data created by the present invention can be automatically stored and a database built of such and other information relating to individual or types of sterilization container. This database may be used to provide estimates of the expected time for safe use of a container. The database may be used to collect and analyze big data.

As used herein the term ‘sterilization container’ may include, but is not limited to a container configured to hold items while they are being sterilized. The term may include a container configured for storing the sterilized items until use of such items. The container may be configured for holding surgical instruments or any other suitable type of instrument, such as but not limited to a medical instrument, a dental instrument, a veterinary instrument, a laboratory instrument and a combination thereof

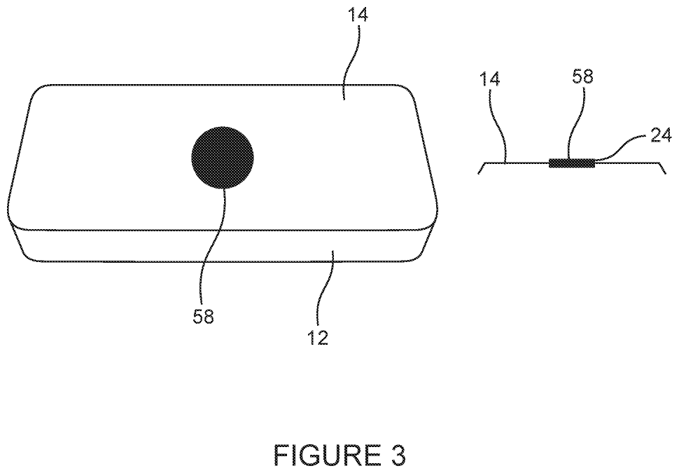

As used herein the term ‘container’ may include, but is not limited to a container featuring a storage tray and a lid, which can be assembled together to create a sealed storage compartment. The sealed compartment may be configured to prevent the contents from being exposed to the external environment. The sealed compartment may be configured to prevent the external environment from being exposed to the contents of the sealed compartment. The container may include one or more inlets for allowing a flow of steam during autoclave sterilization or to allow a flow of any other suitable sterilant into the container. The inlet may be covered with a filter, or with a one-way valve. The container may include a sealing component to seal the inlet. In one or more embodiments, the invention pertains to a container including an inlet covered with a filter and/or a one-way valve and/or with any other suitable cover and a sealing component (such as sealing component 58 of FIGS. 2a and 3).

As used herein the term ‘sealing edge’ may include, but is not limited to the perimeter of openings and the perimeter of the lid of the container, such as, but not limited to a sterilization container. The openings may be openings that can be sealed. The openings may be openings, which are susceptible to damage, resulting in damage to the sealing of the container. The openings may include the opening of the unlidded container and the opening of an inlet or filter or a valve. The sealing edge may include the separation line between the components of the container, such as for example the lid and the storage compartment. The sealing edge when sealed may be designed to block entry of anything from the external environment into the container.

As used herein the term ‘reusable’ may include, but is not limited to a system or parts thereof which can be reused multiple times. For example, the herein containers are meant to be reusable, provided their integrity is maintained. To evaluate the condition of the containers, they may be inspected by the herein methods and systems.

As used herein the terms ‘usability grade’, ‘performance grade’ and ‘sealing grade’ may be used interchangeably and may include, but are not limited to a grade calculated for a container for the level of leakage of light and the ability of the container to maintain sterilization. The usability grade may weigh various variables associated with an inspected container, including, but not limited to dimensions of the container, history of use of the container, date of manufacture, storage conditions, storage duration, expiration date, previous infections associated with previous uses of the container, instruments stored in a container, previous repairs, etc.

As used herein the term ‘connection’ may include, but is not limited to direct and indirect attachment.

As used herein the terms ‘a’ and ‘an’ may mean ‘one’ or ‘more than one’.

As used herein the terms ‘comprising’, ‘including’, ‘containing’, ‘featuring’, ‘having’ and any forms of the terms thereof are inclusive and open ended and do not exclude additional elements or method steps, which are not recited.

The term ‘consisting essentially of’ as used herein means that the scope is limited to the specified elements and those that do not materially affect the basic and novel characteristic(s) of the claimed device and materials.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

All ranges disclosed herein include the endpoints. The use of the term “or” shall be construed to mean “and/or” unless the specific context indicates otherwise.

The term ‘about’, in some embodiments, refers to ±30% of the stated numerical value. In further embodiments, the term refers to ±20% of the stated numerical value. In yet further embodiments, the term refers to ±10% of the stated numerical value.

The principles and operation of a system, such as a detection system, as well as methods of use thereof according to the present invention may be better understood with reference to the figures. The figures show non-limiting aspects of the present invention.

### The Detection System

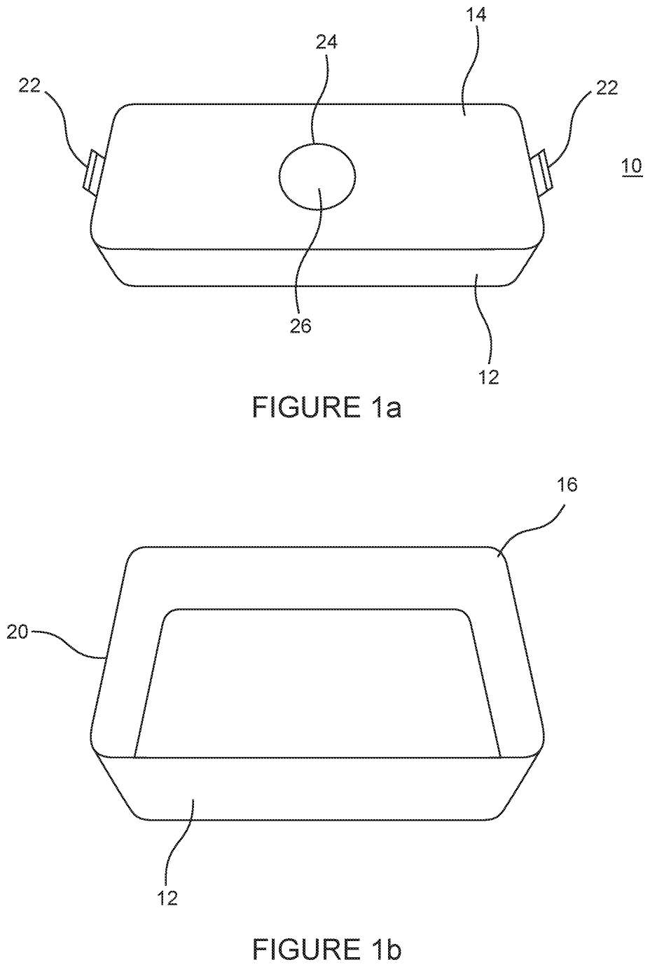

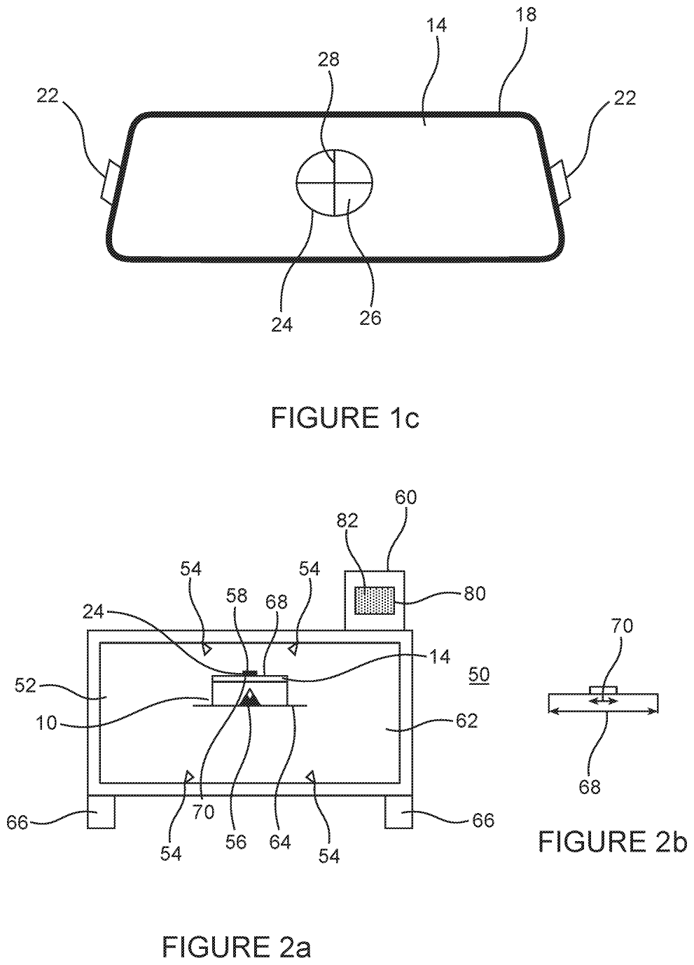

FIG. 1a shows a schematic view of an exemplary sterilization container 10 of the art. The sterilization container 10, includes a body featuring a cavity, which serves as a storage compartment 12. The storage compartment 12 may be configured in any suitable way to hold items to be sterilized, such as but not limited to surgical or medical instruments. The container 10 may include a lid 14. FIG. 1b shows a schematic view of the storage compartment 12 without a lid 14. FIG. 1c shows a schematic view of the lid 14 detached from the storage compartment. The lid 14 may be sized and shaped to fit over the opening 16 of the storage compartment. The opening 16 may be optimally sealed using a sealing means 18, such as, but not limited to a silicone gasket 18 which corresponds to the sealing edge 20 about the opening 16 of the storage compartment 12. In some embodiments, the lid 14 may be secured to the storage compartment 12 using any suitable securing means 22, such as, but not limited to at least one latch 22. The container 10 may include at least one inlet 24 for allowing a flow of steam or any other suitable sterilant into the container 10 during autoclave sterilization. In one non-limiting example the container 10 includes one inlet 24. The inlet 24 is designed to prevent non sterile air, particles, or other contaminants from flowing into the container 10 during storage. The inlet 24 may be covered with a filter 26. Optionally, the inlet 24 is covered with a one -way valve (not shown). The filter 26 may be held in place by a filter holder 28. The sterilization container 10 may be configured for sterilization using any suitable technique, such as, but not limited to sterilization using at least one of steam, plasma, ethylene oxide and gamma radiation. The sterilization containers 10 may be of any suitable size and shape and may be made from any suitable material, which is inert to the conditions of the sterilization procedure, such as, but not limited to aluminum. The sterilization container 10 used in the present invention is made from an opaque material, which does not allow light through its walls. The sterilization container 10 may include identification information such as, but not limited to an identification number. The identification information may include at least one of the name of the manufacturer of the container, the type of container and the date the container was produced. The identification number may be a barcode. The sterilization container 10 may be a single use container or a reusable container.

The sterilization container 10 may be inspected visually before use or at any other suitable time to ascertain the sealing capability of the container 10 and the resulting ability to maintain sterility of contents, which have been sterilized and are stored in the container.

FIGS. 2a and 2b show a schematic view of an exemplary system 50 for inspecting and detecting damage to the sealing ability of a container, such as a sterilization container according to an aspect of the present invention. The damage may be any suitable damage, such as, but not limited to physical damage or mechanical damage. The system may include a chamber 52, at least one light detection device 54 and at least one light source 56. The system 50 may also include a sealing component 58 to seal any inlet 24 of a container 10, such as that of a sterilization container 10. The system 50 may further include a computer 60 with software to run the system.

The system 50 includes a chamber 52. The chamber 52 features a cavity or internal space 62 as shown schematically in the figure. The chamber 52 is configured so that it can be sealed. The chamber 52 may be sized for placement of at least one sterilization container 10 within the chamber 52. In some embodiments, the chamber 52 is sufficiently big for accommodating more than one sterilization container 10. The sterilization container 10 may be placed on the bottom of the chamber 52. Alternatively, or additionally, the chamber may include a shelf or platform 64 for accommodating the sterilization container 10. The shelf 64 may be positioned to fix the sterilization container 10 placed thereon at the correct distance from components of the system 50, such as the light detection components 54. The shelf 64 may be positioned above or below the light detection devices 54, such as cameras. The sterilization container 10 is positioned so that the light detection devices 54 can view the entire perimeters of the lid 14 and/or the inlet 24. The sterilization container 10 may be positioned so that the light detection devices can view the sealing edges 20 of the sterilization container 10. The sterilization container 10 is positioned so that the light detection devices 54 are in a line of sight with the sterilization container 10. The chamber 52 is constructed in any suitable shape, such as, but not limited to a box shape. The walls of the chamber 52 are configured to block light from the outside environment from the field of view of the light detection devices 54 so that the only light detected is from the light source 56. The walls of the chamber 52 may be opaque in order that external light cannot enter the chamber 52, which may interfere with the reading of the system. The chamber 52 may be made from any suitable opaque material, such as, but not limited to metal, plastic or wood. When the material is not opaque, it can be coated with a layer of an opaque substance, such as, but not limited to paint, to prevent light from outside the chamber 52 entering the chamber 52. The system 50 may be configured in any suitable way so that the chamber 52 can sit on a surface. In one embodiment, the system 50 includes legs 66 attached to the chamber 52 for standing the chamber 52 on a surface.

The system 50 includes at least one light detection device 54. The at least one light detection device 54 may be any suitable light detector 54, such as but not limited to at least one of a camera, a video camera, a photodetector and a light sensor. The at least one light detection device 54 may be attached to the walls of the chamber 52 so that it can view a sterilization container 10 placed in the chamber 52. The at least one light detection device 54 may be attached so that it is rotatable. The at least one light detection device 54 may be attached so that it can be moved vertically and horizontally. In some embodiments, the at least one light detection device 54 may be attached and configured to view the perimeter 68 of the lid 14 and optionally the perimeter 70 of the at least one inlet 24 of the sterilization container 10 (FIG. 2b). The at least one light detection device 54 may be attached and configured to view the sealing edge/s 20 of the sterilization container 10. The at least one light detection device 54 may be positioned such to allow visualization of the entire face of the container 10, and/or the entire perimeter 68 and/or 70. Optionally, two, three, four, or more light detection devices 54 are disposed in the chamber 52. Areas of the sterilization container 10 which are susceptible to damage resulting in a breach of sealing of the container 10 include the perimeter 70 of the at least one inlet 24, the perimeter 68 of the lid 14 and the perimeter of the sealing edge 20 of the container. As such these areas are inspected by the system 50 of the present invention. The at least one light detection device 54 may be a plurality of light detection devices 54. In one embodiment, the system 50 may include four light detection devices 54. A light detection device 54 such as a camera 54 may be configured to detect light and also provide an image of the location where light has leaked out from the sterilization container 10. The at least one light detection device 54 may be connected to a computer 60. Connection may be wireless. The at least one light detection device 54 may be controlled manually or may be controlled by the computer 60. Data including images collected by the light detection devices 54 may be received by the computer. Data from the at least one light detection device 54 may show a breach in the sealing of the sterilization container 10 being checked, or may show that the sealing is intact.

The detection system includes at least one light source 56. The at least one light source 56 may be sized for placement in a closed sterilization container 10. In some non-limiting examples, the light source may have a width of up to about 9 cm, a length of up to about 20 cm and a height of up to about 5 cm. The light source 56 is configured for illumination of the inside of the sterilization container. The light source may be any suitable light source, or source of energy (e.g., heat, sound, radiation), which cannot penetrate through the walls of the container and can only leak through a gap or opening of the container. The light source may be any suitable energy source which can be detected by a suitable detection device. One non limiting example of a suitable light source is a LED light. The light source 56 may provide any suitable color of light, such as but not limited to a green led light. The system 50 of the invention may control the color and change the color according to requirement. In some embodiments, the light source 56 may be activated manually. In some embodiments, the light source 56 is illuminated when commanded by the system 50. The at least one light source 56 may be connected wirelessly to the system's computer 60. The system 50 may indicate using any suitable indicator when the light source 56 is on inside the sterilization container 10. The light from the light source 56 in the sterilization container 10 can only be detected by the at least one light detection device 54 when the sealing of the sterilization container 10 is damaged. In a working system no light detected by the light detection device 54 is verification of optimal sealing. The light source 56 may be powered by any suitable power source. In some embodiments, the at least one light source is battery powered. The battery may be a rechargeable battery. The battery power may be set within an acceptable range for providing sufficient power to the light source. The battery power may be controlled by the system to give sufficient intensity and stable lux values. The battery power may be monitored by the system of the present invention. The light source 56 may be set to have the same brightness when used, in order to provide reproduceable results in a plurality of detection systems 50 of the present invention and when using a plurality of different light sources 56 of the present invention. In one non-limiting example the light source may include a unique identification chip, which can be identified by the system. The system may then report an error if a non-approved light source is used and may not perform the test.

The system may include a component to interact with the light. Interaction with the light may include, but is not limited to amplification, reduction, diffusion, filtering or deflection of the light from the light source in order to facilitate the reception by the light detection device. Non limiting examples of such a light interacting component include at least one of a filter, a lens, a diffuser, a reflector and a mirror.

The sterilization container 10 may include at least one inlet 24 as shown in FIG. 1a and FIG. 1c. The at least one inlet 24 may be covered with a filter 26. The at least one inlet 24 may be located in any suitable position on the container 10. In one non-limiting embodiment, the at least one inlet 24 may be positioned on the lid 14 of the container 10. The system 50 of the present invention may include a sealing component 58 to cover the at least one inlet 24 during the inspection process as shown schematically in FIG. 3. The sealing component 58 may be placed over the filter 26. In some embodiments, the filter 26 is removed and the sealing component 58 replaces the filter 26 on the inlet 24 of the sterilization container 10. The sealing component 58 may be fixed in place with a filter holder 28. The sealing component 58 may be any suitable device to sufficiently cover the inlet 24, such as but not limited to an opaque sealing foil 58 or a sealing plug 58. The sealing component 58 may be of a standard size with a similar shape to fit the standard size of the inlets 24 of sterilization containers 10. In some embodiments, the sealing component 58 may have a similar shape and dimensions as the filter 26. In some embodiments, the sealing component 58 may have larger dimensions than the filter 26.

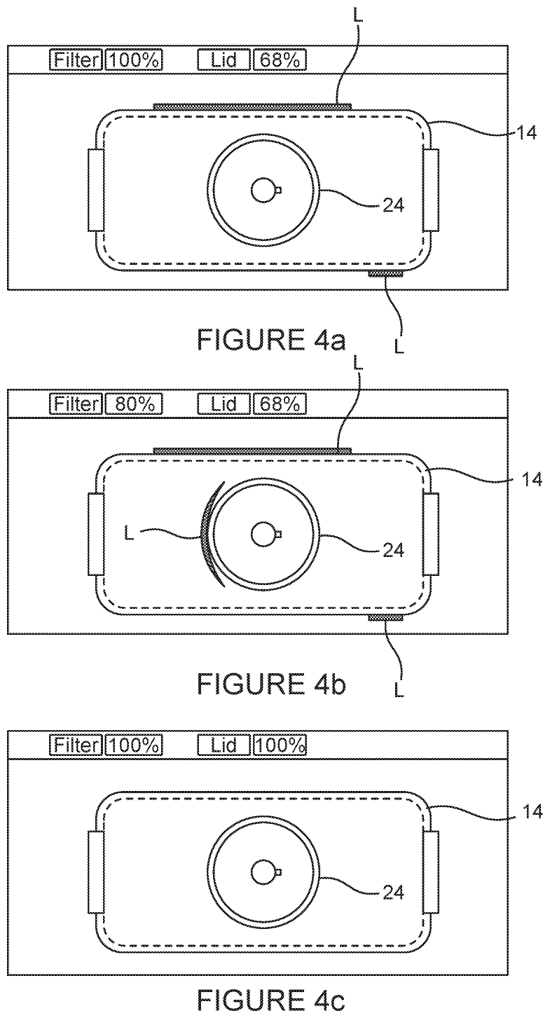

The detection system 50 may include a computer 60. The computer 60 may include image processing software and may feature software for controlling the operation of the detection system 50. The system may include a screen 80 with a display 82. The computerized system may control the at least one light source 56 for activating it and turning it off. The software may include sequences of turning the at least one light source on and off. The software may command the at least one light source 56 to perform a sequence of operations to assist in identifying a leak, such as but not limited to changing illumination power, changing led color to find the wavelength best detected by the light detector and switching the light source on and off. The software may check the at least one light source 56 is working. The computer 60 may control the at least one light detector 54. The computer 60 may monitor any malfunctions in the at least one light detector 54. It may be able to change the position of the at least one light detector 54. The computer 60 may store images and data from the at least one light detector 54. Non limiting examples of images showing leakage of light can be seen in FIGS. 4a, 4b and 4c. FIG. 4a shows leakage of light (L) from the edges of the opening of the container. FIG. 4b shows leakage of light (L) from the edges of the opening of the container and from the inlet 24. FIG. 4c shows no leakage of light (L) from the container. Leakage of light is indicative of damage to the sealing of the sterilization container and a possible contamination as a result of such damage. The software may provide a usability grade which takes into account the sealing capability of a sterilization container being checked. The usability grade of a sterilization container may be based on the amount of light detected by the system. In one non limiting example the usability grade calculates a percent of the perimeter of the opening of the container which is not sealed. For example, a leakage of 32% (i.e., 68% sealing) was calculated for the lid 14 in FIG. 4a. The leakage of the lid 14 was determined by calculating the percentage of the perimeter of a detected area of light leakage with respect to the total perimeter of the lid 68. For example, a leakage of 32% (i.e., 68% sealing) was calculated for the lid 14 and a leakage of 20% (i.e., 80% sealing) was calculated for the inlet 24 in FIG. 4b. The inlet 24 leakage was determined by calculating the percentage of the perimeter of a detected area of light leakage through inlet 24 with respect to the total perimeter 70 of the inlet 24. The software may include different options for data management. The stored data may include identification data, data relating to all inspections performed, such as operator, dates, times and results. An operator may edit data and may view the data and history of a sterilization container. The software may provide different display options, including images of the container and processed images of the container.

The detection system 50 and its components are reusable with any suitable container, such as a sterilization container 10. The at least one light source 56 and the sealing device 58 can be removed from a sterilization container 10 after it is checked and can then be inserted into another sterilization container 10. The system 50 can be used for inspection of one sterilization container 10 at a time. In some embodiments, the system 50 can be used for simultaneous inspection of more than one sterilization container 10. In such embodiments, at least one light source 56 is placed in each sterilization container 10 and at least one light detecting device 54 is associated with a different sterilization container 10 and is attached to the chamber 52 for a clear view of the corresponding associated sterilization container 10.

The sealing inspection system 50 of the present invention can be used by any suitable user. It may be used to inspect any suitable container. The container may be a sterilization container or may be any other container for which optimal sealing is important and the functionality and condition of the container is critical. The system may be used routinely. Non limiting examples of circumstances where use of the system of the present invention is desirable include, a container which is suspected of being damaged and for which visual inspection is not conclusive, a container near its liability expiration date, investigation of infection associated with instruments sterilized in a container, a container which has been serviced, a container which has been stored for a long time and a container which has been repaired.

In some embodiments, the system 50 of the present invention may be used for inspection of the sealing of a container, which is not a sterilization container. The system 50 of the present invention may be substantially the same as described hereinabove for inspection of a sterilization container. A container, which is used to store a sensitive material may not include an inlet. In such an example the system may not include a sealing component 58, however, the other components are the same. In order to avoid repetition, the components of the system 50 will not be described again.

### Method of Checking the Extent of Sealing of a Sterilization Container

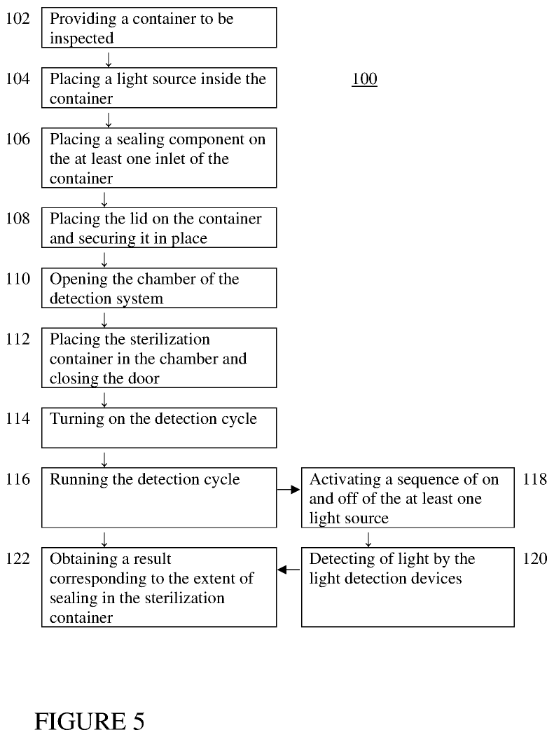

The present invention provides a method of inspecting containers for checking the extent of sealing of a sterilization container 100 as shown schematically in FIG. 5. The order of the steps is not meant to be limiting and any suitable order may be used. The inspection may be performed in any suitable location. The inspection may be done at the same place the sterilization containers are packed with instruments for sterilization. In some embodiments, initially a sterilization container may be manually evaluated by a visual inspection. Such a manual evaluation may be done according to the manufacturer instructions and according to routine inspection procedures as defined by the facility using the containers, such as a hospital. In some embodiments, a container, which does not pass the manual visual inspection is not inspected by the system of the present invention. The system of the present invention may be typically used for inspection of a container which has been approved by manual visual inspection. A sterilization container is provided for inspection 102. The sterilization container is prepared for use with the system of the present invention. The sterilization container may be opened and at least one light source may be placed inside the storage space of the container 104. In some embodiments, at least one light source may be placed inside the container. A sealing component may be placed on the inlet of the container 106. In some embodiments, the filter or valve may be removed. Removing the filter or valve may include removing the filter/valve holder and taking the filter/valve out. A similarly shaped opaque sealing component may be placed over the inlet and the filter holder reattached to hold the sealing component in place over the inlet. In some embodiments, the filter/valve is not removed, and the sealing component may be placed over the filter/valve. The sealing component may be a piece of foil or a foil plug. The sealing component may be sized to fit the standard sized inlet. The lid may be put back on the container and secured to it to close the container 108. In one non limiting examples latches are used to lock the lid onto the opening of the container. The chamber of the system of the present invention may be opened 110. In some embodiments, the front side of the chamber is a door, which is opened. The sterilization container with the inserted at least one light source may be placed in the chamber 112. In some embodiments, the chamber may include a shelf or any other suitable platform on which the sterilization container can be placed. The shelf may be positioned so that the at least one light detection devices, such as cameras, which are attached to the inside of the chamber are optimally placed in relation to the sterilization container placed on the shelf for viewing any light leakage from the sterilization container. The chamber door may be closed resulting in the chamber interior being devoid of light from the outside. In some embodiments, the system is computer controlled. The operator of the system may activate the system to start the inspection process 114 and the process is run 116. The system orders the light source to operate, which may include a sequence of turning the light on and off 118. Light may be initially turned on to check that the system is working. The light detection device can measure the light in the chamber when the light source is turned on. The light detection device can measure the light in the chamber when the light source is turned off. The measurement of the light in the chamber when the light source is turned off may provide a background reading for no light leakage, which can be used for calibration. The light detection devices detect if there is any light 120 and the results are obtained by the system. 122. When the sterilization container is optimally sealed this may be indicated by at least one of no light illumination, light illumination below a predetermined standard level from the sterilization container into the chamber, no detection of light by the at least one light detection device and detection of light below a predetermined level. The inspection process may be very quick and may take up to a few minutes. The results may be processed. Processing may include calculations. The results may include images. The images may be processed. The results may include a scale of how well the container is sealed and this may be based on the amount/intensity of light detected by the one or more light detection devices. The scale may be numerical. The system may include an algorithm for creating a container performance grade. The sterilization container may be approved or disapproved for use according to this performance grade. The sterilization container can be removed from the chamber when the inspection is completed. The at least one light source may be removed from the inside of the sterilization container and the sealing component may be removed from the at least one inlet. In an embodiment where the filter was removed, the filter may be replaced.

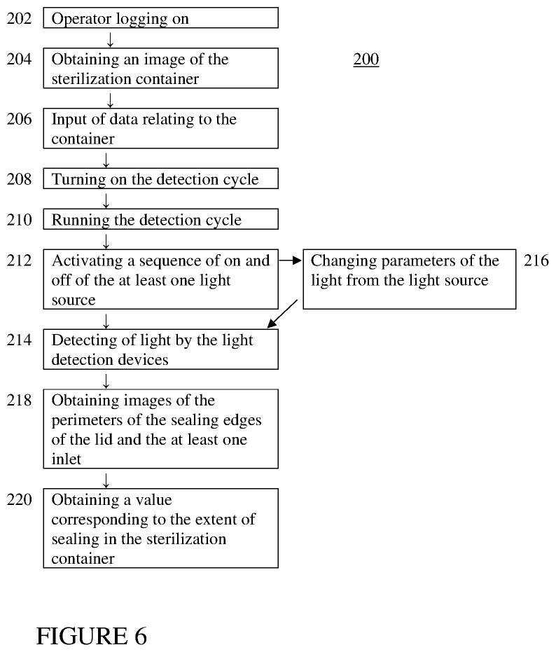

FIG. 6 shows schematically steps that may be included in the running and operation of the system 200. The order of the steps is not meant to be limiting and any suitable order may be used. A user may log on to the computer program 202 of the system. In some embodiments, an image of the container may appear on the screen 204. An identification number may be associated with the container, and this may be inputted by the user in any suitable way 206. The computer may include other information relating to the container. The process may be started in any suitable way, such as, but not limited to by pressing a start button 208. The detection program may be run 210. The light source may be turned on. The system may order the light source to operate in a predefined sequence of turning on and off the light 212. The light detection devices may monitor 214 the chamber for detection of light leaking through the lid sealing edge and inlet. The software may change parameters of the light source and the light detection devices according to the data 216. Parameters may include, but are not limited to the intensity of the light sources, the color of the light sources, the height of the shelf and the angle of the light detection devices. The software can create an image of the perimeters and the leaking areas of the sterilization container 218. The images can be processed in any suitable way. The images can be stored and can be printed. The software can obtain a value corresponding to the extent of the sealing in the sterilization container, which relates to the level of leakage of light from the sterilization container into the chamber 220. The software can calculate a score for a sterilization container. The score may be a usability/performance grade. The score may be based on the percentage of the perimeter of the detected leaking areas of the sealing edge from the total perimeter of the sealing edge. The score may be based on at least one of any suitable parameters that may correlate with the performance of the container. Non limiting examples of suitable parameters include light intensity, location of the leak, and degree of change from the last inspection. The software can calculate a similar score for the perimeter of the inlet. One percentage value may be calculated for the container opening edge and a different percentage value may be calculated for the inlet edge. The data may be printed onto a sticker which can be placed on the sterilization container. The information can be used to repair any damage to the sealing of the container.

The grade provided by the system may be used to monitor and/or determine the ability to maintain sterilization in storage for determining the time a container may be kept in storage without contamination of the sterilized contents of the container. The grade may be defined as the calculated probability of a container to maintain sterilization over the defined shelf life of the container.

The grade may be calculated based on a plurality of different variables that together may influence the ability of the container to maintain sterilization. The algorithm may use Artificial Intelligence techniques to improve accuracy of the grade based on big data and reports from the user on the performance of the container. For example, the algorithm may take into account that damage in a specific location may be more significant than damage in another area. The algorithm may consider that an incremental change in the condition of the container may be calculated differently than a big change in the condition. The calculation process may use a decision making chart. The grade may also be influenced by parameters that are external to the container. External grade influencing parameters may include, but are not limited to last/first containers batch inspected in shift, time of the day/day of the week at which the container was inspected, number of previous sterilization cycles, intensity of use and weight of instruments.

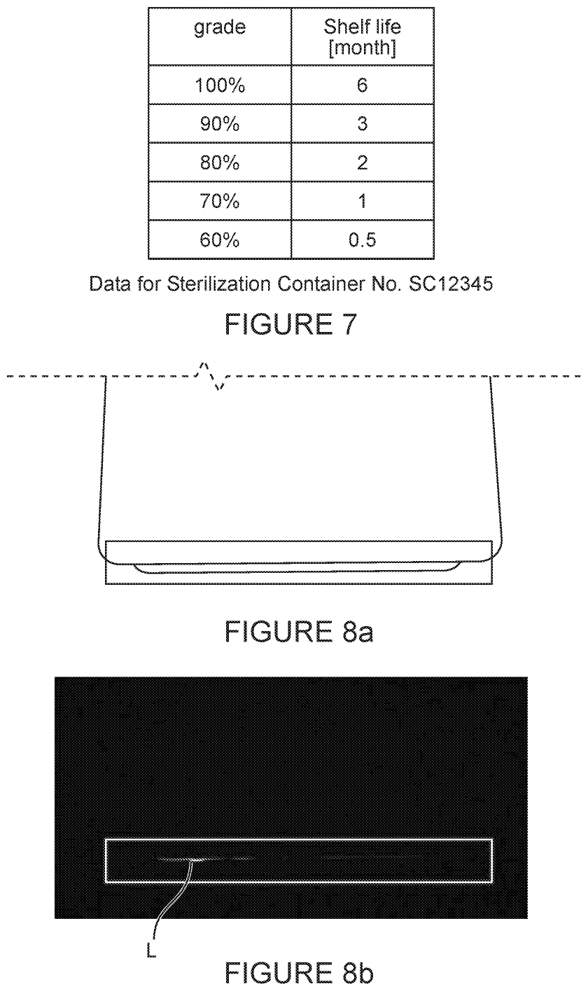

FIG. 7 shows a schematic image of a non-limiting example of using the data of a sterilization container No. SC12345 in such a way. The grade may be used for approving the sterilization container for use according to the level the sterilization container is sealed. A grade of 100 percent means that the entire length of the container opening edge is sealed. For example, a container with a grade of 100 is safe for a shelf life of X months, which in the non-limiting example shown in FIG. 7 is six months. When the grade is less than 100, the approved shelf life may be reduced. For example, a grade of 90 may be safe for storage of Y months, where Y is less than X and which in the non-limiting example shown in FIG. 7 is three months. The system may calculate other variables that may influence the ability to maintain sterilization as described hereinabove.

A calibration test may be conducted on predefined sterilization containers to establish the time for safe use of a specific grade of a sterilization container. The calibration test may be part of the system installation in a medical facility. The sterilization containers may be sterilized with microbial indicators inside each container. The indicators may be checked for microbial growth in predetermined time intervals to establish the time intervals that are safe for use. The same sterilization containers may be inspected by the system of the present invention and given a grade by the system. The data from both tests can then be combined and used to establish the time interval that is safe for a specific grade in a specific facility set up. The system may use this information to recommend a shelf life of a sterilization container. Optionally, a calibration test using microbial indicators may be additionally or alternatively conducted to determine the degree of contamination with respect to the intensity of light and/or accumulated light that is leaking from containers.

The system of the present invention may be used to collect data relating to the history and performance of a sterilization container in terms of sterility. It can be used to create a performance record for a container. Any data associated with a sterilization container can be stored by the system. Data may include, but is not limited to repairs, the level or grade of the sealing of the sterilization container, manufacturing date, infection related to items stored in the container, sterilization conditions, storage conditions, adverse events reported by a user, infection of a patient using instruments stored in the sterilization container, time limit factors, expiry date, time in storage, the number of times the sterilization container has been stored, transportation, the number of times the sterilization container has been sterilized, and data relating to similar containers. Data from different medical facilities can be stored by the system. This data may assist in risk management to prevent infection. The system may also alert users to a sterilization container when it is past its expiry date.

The same method as described hereinabove for a sterilization container, can also be used to ascertain the sealing of a container, which is not a sterilization container.

Reference is made to the following example, which together with the above descriptions illustrates the invention in a non-limiting fashion.

### EXAMPLE 1

A sterilization container was selected. Before loading the container with surgical instruments for sterilization, the sealing of the sterilization container was checked. An LED light source was placed in the sterilization container and the filter was replaced with a piece of foil. The lid was secured to the sterilization container and the sterilization container was placed on a platform in a chamber of the system of the present invention. The system was turned on and a sequence of commands resulted in the light source illuminating the interior of the sterilization container. Cameras which were spaced and attached in the chamber to view the sterilization container were used to detect any light from the light source to indicate if the sealing was breached or not. If the sterilization container was damaged, light would radiate from the inside of the sterilization container into the chamber and the light would be detected by the light detector. FIG. 8a shows schematically a segment of the container being inspected. FIG. 8b shows the segment captured by the camera before image processing. It can be seen that light had leaked through the sealing lip, indicating damage to the sealing lip. The system then calculated a usability grade for the duration of safe storage based on the severity of the leak and other parameters. Based on the data the operator decided to reject the container and send it for repair. The workshop was supplied with the images created by the system of the present invention in order to indicate the exact location and extent of the damage for better mending of the container.

One skilled in the art can appreciate from the foregoing description that the broad systems, devices, and techniques of the aspects of the present invention can be implemented in a variety of forms. Therefore, while the aspects of this invention have been described in connection with particular examples thereof, the true scope of the aspects of the invention should not be so limited since other modifications will become apparent to the skilled practitioner upon a study of the specification, and following claims.

1.-44. (canceled)

45. A system for inspection of containers, the system comprising:
a chamber for accommodating a container, the chamber unlit and sealed to any external light;
at least one light source configured to be positioned in the container to illuminate the interior of the container; and
at least one light detection device disposed at the interior of the chamber and configured to detect light from the at least one light source, wherein the detected light is emitted from the container into the chamber; wherein the detected light is used to detect damage to the container.

46. The system of claim 45, wherein the container is a sterilization container made from an opaque material, the sterilization container comprises
a body comprising a cavity for holding an item to be sterilized;
a lid for covering the cavity;
at least one inlet for allowing flow of a sterilant into the container, the inlet is optionally covered with a filter or valve;
optionally a sealing component for sealing the inlet of the sterilization container; and
optionally, wherein the at least one inlet with a filter is positioned on the lid of the sterilization container.

47. The system of claim 45, wherein the at least one light detection device is at least one camera configured to view the container and show the position of where the container is damaged.

48. The system of claim 46, wherein the system further comprises a sealing component for sealing the inlet of the sterilization container, wherein the sealing component is optionally an opaque foil, which covers the inlet.

49. The system of claim 45, comprising a computer to control the system, wherein the computer comprises software to provide a score of the sealing capability of the container.

50. The system of claim 49, wherein the computer is connected wirelessly to control the at least one light detection device and/or the at least one light source.

51. The system of claim 49, wherein the computer commands the at least one light source to perform a sequence of operations, wherein the sequence of operations comprises at least one of turning the at least one light source on, turning the at least one light source off and changing the color of the light from the at least one light source, wherein the light is measured in the chamber when the at least one light source is turned on and when it is turned off.

52. The system of claim 45, wherein the at least one light source is battery operated and is controlled by the system for at least one of sufficient power, intensity and stable lux values.

53. The system of claim 45, wherein when the container is optimally sealed there is at least one of: no light illumination, light illumination below a predetermined standard level from the container into the chamber, no detection of light by the at least one light detection device and detection of light below a predetermined level.

54. The system of claim 46, wherein when the sealing of the container is damaged, light is radiated from the container into the chamber and detected by the at least one camera.

55. The system of claim 49, wherein the computer calculates the level of leakage of light from the container into the chamber.

56. The system of claim 55, wherein the computer assigns a usability grade to the container according to the level of leakage of light, and wherein the container is approved or disapproved for use according to the usability grade, optionally wherein the usability grade comprises a percentage of the perimeter of a detected area of light leakage to the total perimeter of the container or a portion thereof.

57. The system of claim 45, comprising a platform on which the container is placed, wherein the platform is optimally positioned in relation to the at least one light detection device for detecting of the light emitted from the container into the chamber.

58. The system of claim 45, wherein the container comprises a lid that covers the opening of the container and wherein the light detection device detects light emitted from the perimeter of opening of the container.

59. A method of inspecting containers, the method comprising;
providing a system of claim 45;
placing at least one light source in the container, wherein the container is made from an opaque material and comprises a body comprising a cavity for holding an item; and a lid for covering the cavity;
closing the container;
placing the container in the chamber;
closing the chamber;
activating the at least one light source; and
measuring light emitted from the container into the chamber.

60. The method of claim 59, wherein the container is a sterilization container, optionally wherein the sterilization container comprises at least one inlet covered with a filter or valve and wherein the method comprises a step of replacing the filter or valve or covering the filter or valve of the inlet of the container with a sealing component.

61. The method of claim 59, wherein activating the at least one light source comprises activating the light source to ascertain the system is working, and wherein activating the at least one light source comprises turning the light source on.

62. The method of claim 59, wherein measuring light emitted from the container further comprises calculating the level the container is sealed according to the measured light.

63. The method of claim 59, comprising calculating a usability grade, wherein the usability grade is a percentage of the perimeter of a detected area of light leakage to the total perimeter of the container or a portion thereof.

64. The method of claim 63, further comprising approving the container for use according to the level the sterilization container is sealed.
